(19) United States
(12) Patent Application Publication
(10) Pub. No.: US 2026/0098006 A1
(43) Pub. Date: Apr. 9, 2026

(54) SYSTEMS FOR PURIFYING ACETONE

(71) Applicants: Eric Wing-Tak Wong, Houston, TX (US); Truc Van Vu, Houston, TX (US)

(72) Inventors: Eric Wing-Tak Wong, Houston, TX (US); Truc Van Vu, Houston, TX (US)

(73) Assignee: KELLOGG BROWN & ROOT LLC, Houston, TX (US)

(21) Appl. No.: 19/349,753

(22) Filed: Oct. 3, 2025

Related U.S. Application Data
(60) Provisional application No. 63/704,497, filed on Oct. 7, 2024.

(51) Int. Cl. C07C 45/82; B01D 3/00; B01D 3/14; B01D 3/34

(52) U.S. Cl. CPC C07C 45/82; B01D 3/009; B01D 3/143; B01D 3/34

(57) ABSTRACT

A system and method for purifying acetone from a first feed stream and a second feed stream. The system may include a first distillation column for receiving the first feed stream. A second distillation column may be in fluid communication with the first distillation column. The second distillation column may include a caustic inlet to treat contents of the second distillation column with caustic, and outputs purified acetone. A third distillation column may be in a fluid communication with the second distillation column. A caustic treatment vessel may be in fluid communication with the second distillation column and provides a caustic treatment. The first distillation column can be in direct fluid communication with the second feed stream, the second distillation column can be in fluid communication with the second feed stream, and/or the caustic treatment vessel can be in fluid communication between the caustic inlet and the second feed stream.

### CROSS REFERENCE TO RELATED APPLICATION

This application claims priority to U.S. Provisional Patent Application having Ser. No. 63/704,497 filed on Oct. 7, 2024 which is incorporated by reference herein

### TECHNICAL FIELD

The present disclosure relates to systems for and methods of purifying acetone from a feed stream.

### INTRODUCTION

Acetone is a compound used in many chemical reactions as a solvent and/or reactant. Acetone can be prepared by various chemical reactions. Numerous other compounds are soluble in acetone and such compounds can be separated from acetone by various ways.

### SUMMARY

The disclosure includes a system for purifying acetone from a first feed stream including acetone, cumene, phenol, alpha methyl styrene, water and methanol, and a second feed stream rich in acetone and including aldehydes and methanol. The system may include a first distillation column including a first feed stream inlet to receive the first feed stream, a first distillation column top outlet to output an acetone enriched fluid, and a first distillation column bottom outlet to output a phenol enriched outflow.

The system may include a second distillation column including a second distillation column acetone enriched inlet in fluid communication with the first distillation column top outlet, a caustic inlet to treat contents of the second distillation column with caustic, the caustic inlet being positioned above the second distillation column acetone enriched inlet, an acetone product outlet to output purified acetone, the acetone product outlet being positioned above the caustic inlet, a side-draw outlet positioned below the acetone product outlet, a second distillation column top lights outlet to output a first volatile impurity, the second distillation column top lights outlet being positioned above the acetone product outlet, and a second distillation column bottom outlet to output phenate.

The system may include a third distillation column including a third distillation column inlet in fluid communication with the side-draw outlet, a third distillation column top lights outlet to output a second volatile impurity, and a third distillation column bottom outlet.

The system may include a caustic treatment vessel in fluid communication between the first distillation column top outlet and the caustic inlet, in fluid communication between the second feed stream and the second distillation column, in fluid communication between the side-draw outlet and the third distillation column inlet, or in fluid communication between the third distillation column bottom outlet and the caustic inlet of the second distillation column.

In the system, the first distillation column may include a first distillation column auxiliary inlet in direct fluid communication with the second feed stream, the second distillation column may include a second distillation column auxiliary inlet in fluid communication with the second feed stream, and/or the caustic treatment vessel may be in fluid communication between the caustic inlet and the second feed stream.

The disclosure includes a method of purifying acetone from a first feed stream including acetone, cumene, phenol, alpha methyl styrene, water and methanol, and a second feed stream rich in acetone and including aldehydes and methanol. The method may include distilling the first feed stream in a first distillation column such that a phenol enriched outflow exits from a first distillation column bottom outlet and such that an acetone enriched outflow exits from a first distillation column top outlet.

The method may include distilling the acetone enriched outflow in the presence of a caustic fluid such that methanol exits out of a second distillation column top lights outlet, such that a purified acetone stream exits out of an acetone product outlet positioned below the second distillation column top lights outlet, such that an impure acetone stream exits out of an impure acetone outlet positioned below the acetone product outlet, and such that a crude stream exits out of a side-draw outlet positioned below the impure acetone outlet.

The method may include distilling the crude stream in a third distillation column such that methanol exits out of a third distillation column top lights outlet and such that a bottom outlet fluid exits out of a third distillation column bottom outlet where the bottom outlet fluid may be contacted with a caustic to form a caustic treated bottom outlet fluid and the caustic treated bottom outlet fluid may be provided to the second distillation column, or contacting the crude stream with a caustic to form a caustic treated crude stream and distilling the caustic treated crude stream in the third distillation column such that methanol exits out of the third distillation column top lights outlet and such that the bottom outlet fluid exits out of the third distillation column bottom outlet wherein the bottom outlet fluid may be provided to the second distillation column.

The method may include providing the second feed stream directly to the first distillation column, providing the second feed stream directly to the second distillation column, or caustic treating the second feed stream to form a caustic treated second feed stream and providing the caustic treated second feed stream directly to the second distillation column.

### BRIEF DESCRIPTION OF THE DRAWINGS

Features and advantages of the present disclosure will be apparent from the following description of various exemplary embodiments, as illustrated in the accompanying drawings, wherein like reference numbers generally indicate identical, functionally similar, and/or structurally similar elements. Arrows between components of the figures are used to indicate the flow direction of fluids within the systems.

FIGS. 1A to 1C are schematic views of acetone purification systems for purifying acetone from a first feed stream and a second feed stream. FIG. 1A shows an acetone purification system with the second feed stream being supplied to a first location. FIG. 1B shows an acetone purification system with the second feed stream being supplied to a second location. FIG. 1C shows an acetone purification system with the second feed stream being supplied to both the first location and the second location.

FIGS. 2A to 2C are schematic views of acetone purification systems for purifying acetone from a first feed stream and a second feed stream. FIG. 2A shows an acetone purification system with the second feed stream being supplied to a first location. FIG. 2B shows an acetone purification system with the second feed stream being supplied to a second location. FIG. 2C shows an acetone purification system with the second feed stream being supplied to both the first location and the second location.

FIGS. 3A to 3C are schematic views of acetone purification systems for purifying acetone from a first feed stream and a second feed stream. FIG. 3A shows an acetone purification system with the second feed stream being caustic treated and supplied to a first location. FIG. 3B shows an acetone purification system with the second feed stream being caustic treated and supplied to a second location. FIG. 3C shows an acetone purification system with the second feed stream being caustic treated and supplied to a third location.

FIG. 4 is a schematic view of an acetone purification system for purifying acetone from a first feed stream and a second feed stream in accordance with an embodiment.

FIG. 5 is a schematic view of an acetone purification system for purifying acetone from a first feed stream and a second feed stream in accordance with an embodiment.

### DETAILED DESCRIPTION

Features, advantages, and embodiments of the present disclosure are set forth or apparent from a consideration of the following detailed description, drawings, and claims. Moreover, the following detailed description is exemplary and intended to provide explanation without limiting the scope of the disclosure as claimed.

Various embodiments are discussed in detail below. While specific embodiments are discussed, this is done for illustration purposes only. A person skilled in the relevant art will recognize that other components and configurations may be used without departing from the present disclosure.

As used herein, a “fluid” is a liquid, a gas, a supercritical fluid, or a combination thereof.

As used herein, a first component is in “fluid communication” with a second component if a fluid can flow along a flow path joining the first component to the second component. The term “fluid communication” does not imply a directionality to the flow of the fluid. That is, the fluid could flow from the first component to the second component or from the second component to the first component. Similarly, a flow path joining the first component to the second component does not imply a directionality to the flow of the fluid and the fluid could flow along the flow path from the first component to the second component or along the flow path from the second component to the first component. If a first component is in fluid communication with a second component, then the first component and the second component are in fluid communication. If a first component is in fluid communication with a second component, then the first component and the second component are joined by a flow path.

As used herein, a first component is in “direct fluid communication” with a second component if the first component and the second component are joined by a flow path that is free from an intervening separation apparatus or reaction apparatus. A separation apparatus is any apparatus used to separate components of the fluid flowing along the flow path. An example of a separation apparatus is a distillation column. A reaction apparatus is any apparatus used to react a component of the fluid flowing along the flow path. An example of a reaction apparatus is a caustic treatment vessel.

As used herein, a third component is in fluid communication between a first component and a second component if the third component is positioned along a flow path that joins the first component and the second component. A third component that is in fluid communication between a first component and a second component is in fluid communication with the first component and the second component.

As used herein, a “top outlet” of a distillation column is an outlet where compounds having the lowest boiling point escape the distillation column. Typically, the top outlet of a distillation column has the lowest temperature of all outlets of the distillation column.

As used herein, a “bottom outlet” of a distillation column is an outlet where compounds having the highest boiling point escape the distillation column. Typically, the bottom outlet of a distillation column has the highest temperature of all outlets of the distillation column.

As used herein, a first inlet or a first outlet of a distillation column is “positioned below” a second inlet or a second outlet of the distillation column if the second inlet or the second outlet is positioned closer to the top outlet of the distillation column than the first inlet or the first outlet.

As used herein, a first inlet or a first outlet of a distillation column is “positioned above” a second inlet or a second outlet of the distillation column if the first inlet or the first outlet is positioned closer to the top outlet of the distillation column than the second inlet or the second outlet.

The terms “positioned below” and “positioned above” are related by symmetry. If a first inlet or a first outlet of a distillation column is positioned above a second inlet or a second outlet of the distillation column, then the second inlet or second outlet is positioned below the first inlet or the first outlet. Similarly, if a first inlet or a first outlet of a distillation column is positioned below a second inlet or a second outlet of the distillation column, then the second inlet or second outlet is positioned above the first inlet or the first outlet.

The terms “positioned below” and “positioned above” are related by ordering. If a first inlet or a first outlet of a distillation column is positioned above a second inlet or a second outlet of the distillation column and the second inlet or the second outlet of the distillation column is positioned above a third inlet or a third outlet of the distillation column, then the first inlet or the first outlet of the distillation column is positioned above the third inlet or the third outlet of the distillation column and the third inlet or the third outlet of the distillation column is positioned below the first inlet or the first outlet of the distillation column.

As used herein, a fluid is “provided” to a component if the fluid flows along a flow path to the component.

As used herein, a fluid is “provided directly” to a component if the fluid flows along a flow path to the component and the flow path is free from an intervening separation apparatus or a reaction apparatus.

The terms “upstream” and “downstream” refer to the relative direction with respect to fluid flow in a fluid pathway. For example, “upstream” refers to the direction from which the fluid flows, and “downstream” refers to the direction to which the fluid flows. In another example, a first component is “downstream” of a second component if fluid is provided from the second component to the first component, and a first component is “upstream” of a second component if fluid is provided from the first component to the second component.

As used herein, a fluid is “enriched” in a compound after a separation process (e.g., a distillation or a caustic treatment) if the fluid has a higher concentration of the compound after the separation process than a fluid entering the separation process.

In a distillation process “lights” are volatile compounds that can be removed from a distillation column as a vapor.

As used herein, a fluid is “rich” in a compound if the compound is present in the fluid at a weight percent by total weight of the fluid that is greater than or equal to the weight percent of any single other compound present in the fluid.

The singular forms “a,” “an,” and “the” include plural references unless the context clearly dictates otherwise.

As used herein, the terms “first,” “second,” “third,” etc., are used for labeling purposes only and do not imply any ordering unless the context clearly dictates otherwise.

Here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other.

As noted above, acetone can be produced by various chemical reactions. For example, phenol and acetone are produced in various processes, the most common of which may be known variously as the Hock Process, the Hock and Lang Process, or the cumene-to-phenol process, among others. This process begins with the oxidation of cumene (isopropyl benzene) to form cumene hydroperoxide (CHP). The CHP may be cleaved in the presence of an acid catalyst to form phenol and acetone. The cleavage product may then be neutralized and separated to recover purified phenol, acetone, and/or alpha-methyl styrene products.

More than 60% of the world's phenol production may be for use in the production of bisphenol A.

Bisphenol A may be produced by the condensation reaction of two moles of phenol and one mole of acetone in the presence of an acid catalyst, such as a cation exchange resin. The reaction product may contain unreacted phenol and acetone. The excess phenol and acetone may then be separated from the bisphenol A product and recycled.

The acetone feed from the phenol plant can contain methanol and aldehydes as impurities. The presence of methanol impurity in acetone acts as a catalyst poison inside the bisphenol-A reactor, thereby shortening the lifespan of the catalyst. The aldehydes impurities in the acetone degrade the bisphenol-A product quality since the aldehydes reacts with other components in the reaction mixture. These side reactions can lead to the formation of colored molecules resulting in more purification expenses to achieve a high-quality BPA product for the production of polycarbonates and high-quality epoxy resins. Thus, the unreacted phenol and acetone in the bisphenol-A plant must be purified to minimize the methanol and aldehydes feeding the bisphenol-A reactor. The purification of recycled acetone in a bisphenol-A plant to remove methanol and aldehydes can result in an acetone-rich stream that contains methanol and aldehydes. The acetone content in this purge stream can be one percent to three percent of the total acetone being fed into the bisphenol-A plant.

Most of the new phenol and bisphenol-A plants built in the world over the last twenty years are typically located within the same vicinity. If the acetone-rich purge stream in the bisphenol-A plant can be processed in the phenol plant and recovered, this process would significantly reduce the cost of producing phenol and bisphenol-A. Therefore, there may be a need to develop a method to improve the acetone recovery and purification process when both phenol and bisphenol-A plants are located within the same facility.

When multiple chemical reactions are performed and multiple feed streams need to be purified, separately purifying each feed stream can be an efficient approach. Each feed stream can have a unique impurity profile, and separate purification of multiple feed streams avoids cross contaminating each feed stream with impurities from the other feed stream. Such cross contamination can increase the complexity and difficulty of subsequent purification processes. Here, however, the inventor discovered that the cumene hydroperoxide cleavage product and the acetone stream from a bisphenol A production plant can be co-purified using the systems and methods disclosed herein with synergistic and unexpected benefits. These synergistic benefits include, for example, reduced energy expenditure to purify acetone from the feed streams and improved purity of the product acetone stream.

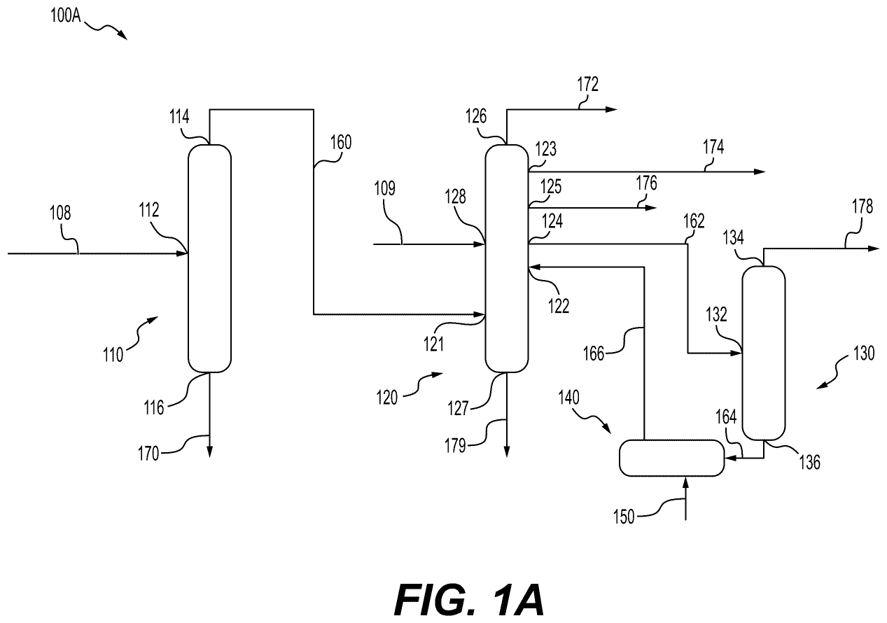

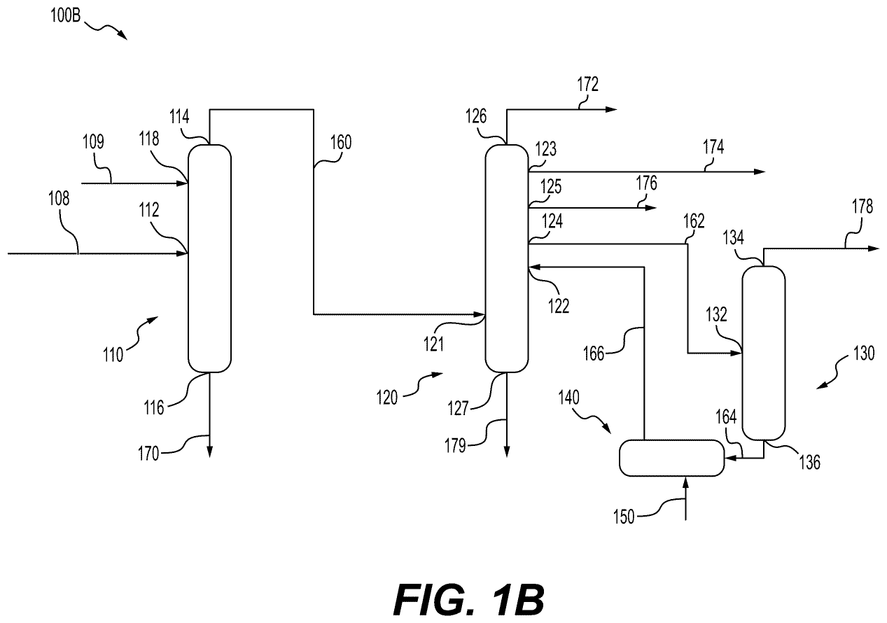

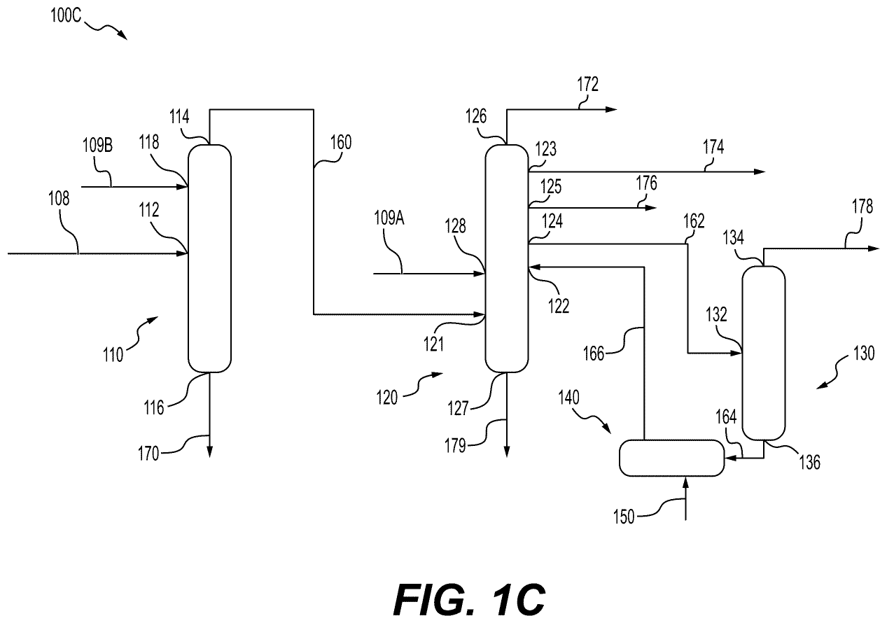

FIG. 1A is a schematic view of an acetone purification system 100A according to an embodiment. The acetone purification system 100A depicted in FIG. 1A can be used to purify acetone from a first feed stream 108 and a second feed stream 109. The first feed stream 108 may include acetone, cumene, phenol, methanol, aldehydes, alpha-methyl styrene (AMS), and water. In some embodiments, the first feed stream may be the cumene hydroperoxide cleavage product or may be derived from the cumene hydroperoxide cleavage reaction. The second feed stream 109 may include acetone, aldehydes, and methanol. In some embodiments, the second feed stream may be the acetone-rich stream from a bisphenol A production process.

The acetone purification system 100A shown in FIG. 1A may include a first distillation column 110. The first feed stream 108 can be provided to the first distillation column 110 via a first feed stream inlet 112. The first distillation column 110 may have a first distillation column top outlet 114, and a first distillation column bottom outlet 116.

The first feed stream 108 can be distilled in the first distillation column 110 such that a phenol enriched outflow 170 exits from the first distillation column bottom outlet 116 and such that an acetone enriched outflow 160 exits from the first distillation column top outlet 114. The acetone enriched outflow 160 can then be provided to a second distillation column 120 in fluid communication with the first distillation column 110.

The second distillation column 120 may include a second distillation column acetone enriched inlet 121 in fluid communication with the first distillation column top outlet 114, and the second distillation column acetone enriched inlet 121 may receive the acetone enriched outflow 160. The second distillation column 120 can include a plurality of inlets and outlets to receive fluids, to discharge fluids, and to treat the fluid in the second distillation column 120. The fluid in the second distillation column 120 can be treated with a caustic treated fluid 166. The second distillation column 120 may include a caustic inlet 122 positioned above the second distillation column acetone enriched inlet 121 to receive the caustic treated fluid 166. Treating fluid in the second distillation column with caustic can remove aldehyde from the process stream by forming heavy ketones via aldol condensation and can deprotonate phenol to form, e.g., sodium phenate 179. The heavy ketones can be removed together with the sodium phenate 179 from the bottom of a second distillation column bottom outlet 127.

The second distillation column 120 may include a second distillation column top lights outlet 126 where low boiling point impurities 172 such as methanol can exit the second distillation column 120. An acetone product outlet 123 may be positioned below the second distillation column top lights outlet 126 and/or may be positioned above a side-draw outlet 124. The acetone product outlet 123 may provide a purified acetone stream 174 that exits the second distillation column 120.

As discussed above, the inventor discovered that the cumene hydroperoxide cleavage product and the acetone stream from a bisphenol A production plant can be co-purified using the systems and methods disclosed herein with synergistic and unexpected benefits. For example, systems and methods can provide the purified acetone stream 174 with a high degree of purity. For example, in some embodiments, the purified acetone has a total concentration of aldehydes less than one hundred fifty parts per million by weight. In some embodiments, the purified acetone has a total concentration of aldehydes ranging from twenty parts per million by weight to one hundred parts per million by weight. In some embodiments, the purified acetone has a methanol content of less than two hundred fifty parts per million by weight. In some embodiments, the purified acetone has a methanol content ranging from fifty parts per million by weight to one hundred parts per million by weight. In some embodiments, the purified acetone stream 174 may have at least ninety-nine weight percent acetone by total weight of the purified steam.

An impure acetone outlet 125 may be positioned below the acetone product outlet 123 and/or may be positioned above the side-draw outlet 124 to outflow an impure acetone exit stream 176 from the second distillation column 120. The impure acetone exit stream 176 may include acetone and, in some embodiments, the impure acetone stream 176 may have a weight percentage of acetone ranging from one weight percent to ninety weight percent by total weight of the impure acetone stream. In some embodiments, the impure acetone exit stream 176 may be recycled to a cumene hydroperoxide cleavage reactor (e.g., the impure acetone exit stream may be in fluid communication with the cumene hydroperoxide cleavage reactor). For example, the impure acetone exit stream 176 may be recycled to the cumene hydroperoxide cleavage reactor that produces feed 108 or from which the cleavage reaction feed 108 may be derived.

In some embodiments, a second feed stream 109 may be supplied to the second distillation column 120. Supplying the second feed stream 109 to the second distillation column 120 may improve energy efficiency of the system as compared to supplying the second feed stream 109 to the first distillation column 110 because the second feed stream 109 may include a higher concentration of lower boiling point compounds than the first feed stream 108 and those lower boiling point compounds would utilize energy to flow out the first distillation column top outlet 114. As depicted in FIG. 1A, for example, the second distillation column 120 may include a second distillation column auxiliary inlet 128 through which the second feed stream 109 can be provided to the second distillation column 120. In the embodiment depicted in FIG. 1A, the second feed stream 109 flows into the acetone purification system 100A via the second distillation column auxiliary inlet 128. In some embodiments, the acetone content of the second feed stream 109 ranges from 1% to 10% of the acetone content of the enriched outflow 160.

As discussed above, the second distillation column 120 may include a side-draw outlet 124 positioned below the acetone product outlet 123 (e.g., positioned below the impure acetone outlet 125) and/or positioned above the second distillation column acetone enriched inlet 121. A crude stream 162 can exit out of the side-draw outlet 124 and into a third distillation column 130. In some embodiments, the crude stream 162 has from 0.1 weight percent acetone to one weight percent acetone by total weight of the crude stream 162. The third distillation column 130 may be in fluid communication with the second distillation column 120. The third distillation column 130 may include a third distillation column inlet 132 in fluid communication with the side-draw outlet 124 to receive the crude stream 162.

The third distillation column 130 may include a third distillation column top lights outlet 134 and a third distillation column bottom outlet 136. Low boiling point impurities 178 such as methanol can exit the third distillation column 130 through the third distillation column top lights outlet 134. Higher boiling point compounds, such as acetone and higher boiling aldehyde impurities, can exit the third distillation column bottom outlet 136, as a bottom outlet fluid 164. The third distillation column bottom outlet 136 may be in fluid communication with the caustic inlet 122 of the second distillation column 120.

In FIG. 1A, a caustic treatment vessel 140 may be in fluid communication with the third distillation column bottom outlet 136 and the caustic inlet 122 of the second distillation column 120. More specifically, the caustic treatment vessel 140 may be positioned downstream of the third distillation column bottom outlet 136 and upstream of the caustic inlet 122 of the second distillation column 120. A bottom outlet fluid 164 may flow out of the third distillation column bottom outlet 136 and into the caustic treatment vessel 140. The bottom outlet fluid 164 that flows into the caustic treatment vessel 140 may be subjected to a caustic treatment within the caustic treatment vessel 140. A duration of the caustic treatment is not particularly limited. For example, the bottom outlet fluid 164 that flows into the caustic treatment vessel 140 may be subjected to a caustic treatment within the caustic treatment vessel 140 for a residence time ranging from three minutes to sixty minutes. The caustic treatment vessel 140 may be free from internal structures or may include one or more of static mixing internals, an agitator, or a circulation pump. Fluid in the caustic treatment vessel may be maintained at a temperature suitable for chemical reactions (e.g., deprotonation reactions and/or heavy ketones formation via aldol condensation) associated with the caustic treatment to occur. In some embodiments, for example, the caustic treatment vessel has a temperature of forty-five degrees Celsius to seventy-five degrees Celsius. Caustic 150 may be fed to the caustic treatment vessel 140 to maintain a caustic pH (e.g., a pH ranging from eight to fourteen, from nine to fourteen, from ten to fourteen, or from eleven to fourteen). Caustic treatment of the bottom outlet fluid 164 in the caustic treatment vessel 140 generates a caustic treated fluid 166 that may be provided back to the second distillation column 120 via the caustic inlet 122 for purification in the second distillation column 120 as discussed above. In this way, the third distillation column 130 and the caustic treatment vessel 140 can form a loop with the second distillation column 120. This loop can increase the purity and yield of the purified acetone stream that exits the second distillation column 120.

The second feed stream 109 can be supplied to the acetone purification system at other locations, and FIGS. 1B and 1C show an acetone purification system 100B and an acetone purification system 100C, each with alternate feed locations for the second feed stream 109. The components in acetone purification system 100B and acetone purification system 100C are otherwise similar to the components depicted in FIG. 1A and the discussion above applies to these systems. Similarly, the fluid streams in FIGS. 1B and 1C may be referred to using the same reference numerals as in FIG. 1A and the discussion above applies to these systems. The fluid streams and components may, however, have different contents and/or different compositions depending on, for example, the arrangement of components in the system, the specific operating parameters used in conjunction with the system, etc.

FIG. 1B is a schematic view of an acetone purification system 100B in which the second feed stream 109 may be supplied to the first distillation column 110 instead of supplying the second feed stream 109 directly to the second distillation column 120. For example, the first distillation column 110 may include a first distillation column auxiliary inlet 118 and the second feed stream 109 may be provided directly to the first distillation column 110 via the first distillation column auxiliary inlet 118. If the first distillation column 110 includes a first distillation column auxiliary inlet 118 and the second feed stream 109 is provided directly to the first distillation column 110 via the first distillation column auxiliary inlet 118, the acetone enriched outflow 160 or the phenol enriched outflow 170 is changed by.

FIG. 1C is a schematic view of an acetone purification system 100C in which the second feed stream 109 may be supplied to both the first distillation column 110 and the second distillation column 120. As depicted in FIG. 1C, the second feed stream can be split into a first portion of the second feed stream 109A and a second portion of the second feed stream 109B. A weight fraction of the first portion of the second feed stream to the 109A divided by a weight of the second portion of the second feed stream 109B can range, for example, from0.1 to 0.99 (or any subrange contained therein). The first portion of the second feed stream 109A can be provided to the second distillation column 120 via the second distillation column auxiliary inlet 128, and the second portion of the second feed stream 109B can be provided to the first distillation column 110 via the first distillation column auxiliary inlet 118.

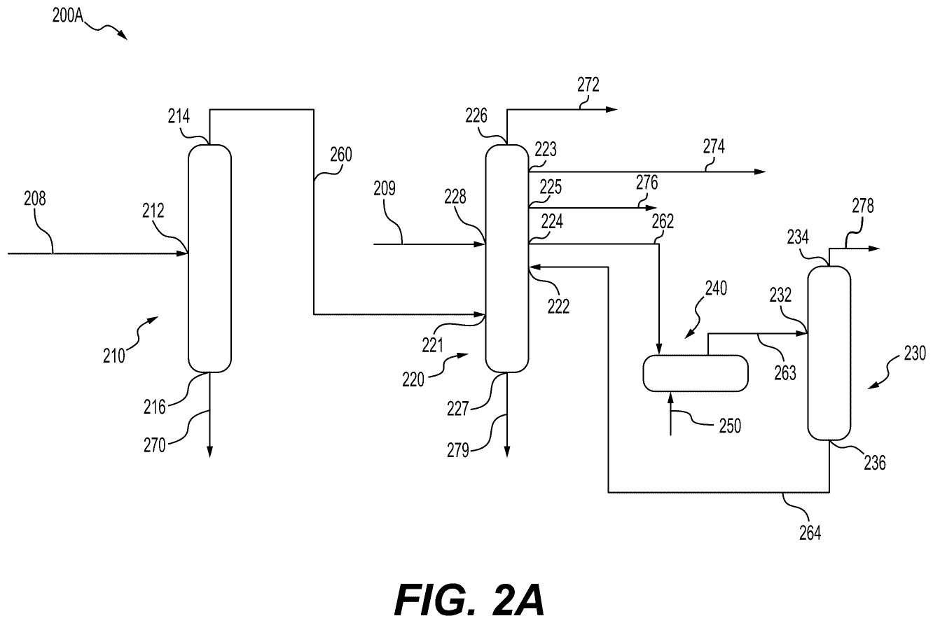

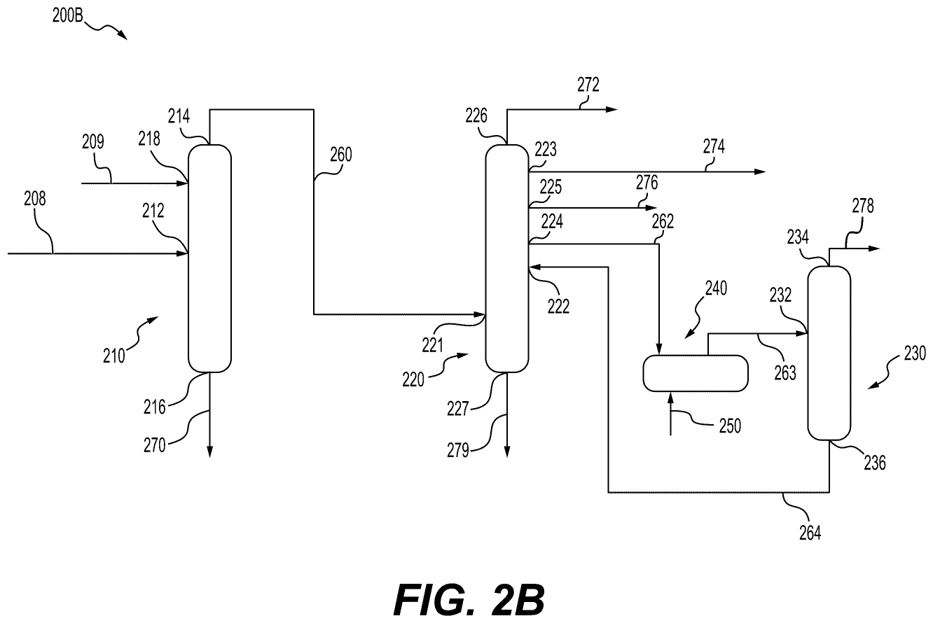

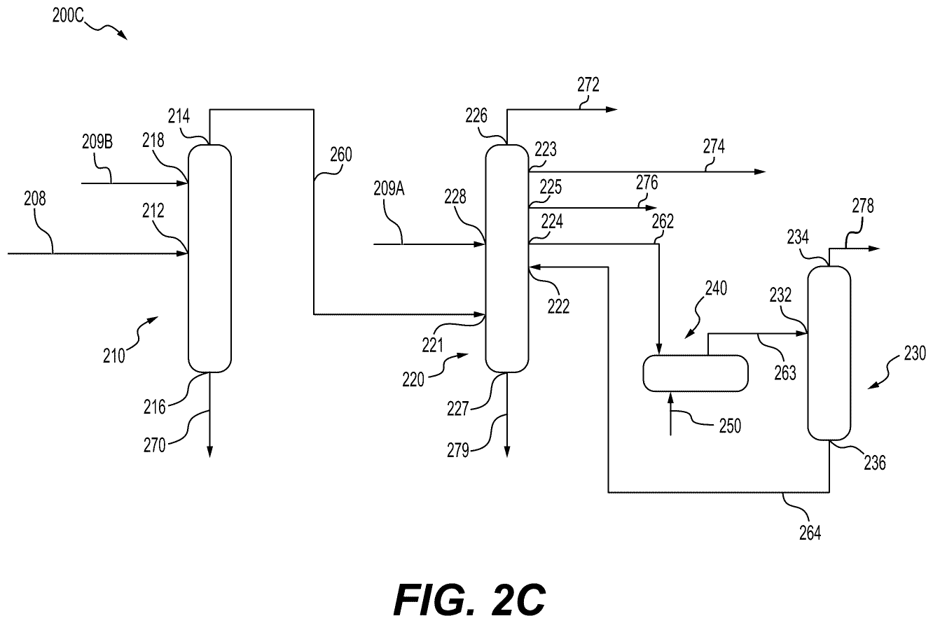

The caustic treatment vessel 140 may be provided downstream the second distillation column 120 and upstream the third distillation column 130 instead of as described with reference to FIGS. 1A and 1B. FIGS. 2A to 2C are schematic views of examples of acetone purification systems for purifying acetone from a first feed stream and a second feed stream with a caustic treatment vessel upstream of the third distillation column. As described earlier with reference to FIGS. 1A to 1C, the second feed system may be provided at different locations independent of the location of the caustic treatment vessel. FIG. 2A shows an acetone purification system with the second feed stream being supplied to a first location. FIG. 2B shows an acetone purification system with the second feed stream being supplied to a second location. FIG. 2C shows an acetone purification system with the second feed stream being supplied to both the first location and the second location.

Several features of FIGS. 2A to 2C are shown and labeled with reference numerals, but not explicitly described herein. Elements of FIGS. 2A to 2C with reference numerals in the 200-series, but not described below, are identical to those corresponding elements in FIGS. 1A to 1C, with otherwise identical reference numerals in the 100-series except the fluid streams and components may have different contents and/or different compositions depending on, for example, the arrangement of components in the system, the specific operating parameters used in conjunction with the system, etc.

FIG. 2A is a schematic view of an acetone purification system 200A in which the caustic treatment vessel may be provided upstream of the third distillation column. The acetone purification system 200A may be similar to the acetone purification system 100A depicted in FIG. 1A other than for the location of a caustic treatment vessel 240. The caustic treatment vessel 240 may be similar to the caustic treatment vessel 140 discussed above. As shown in FIG. 2A the caustic treatment vessel 240 may be positioned upstream of the third distillation column inlet 232 such that a caustic treated fluid 263 is provided to the third distillation column 230. As illustrated in FIG. 2A, the caustic treatment vessel 240 may be positioned between the side-draw outlet 224 of the second distillation column 220 and the third distillation column inlet 232, downstream of the side-draw outlet 224. The caustic treatment vessel 240 receives the crude stream 262 from the second distillation column 220 via the side-draw outlet 224. The crude stream 262 may be treated with caustic 250 in the caustic treatment vessel 240 to generate the caustic treated fluid 263 that may be provided to the third distillation column 230. The caustic treated fluid 263 may contain acetone, water, heavy ketones formed by aldol condensation of aldehydes, and/or deprotonated impurities. Separating the caustic treated fluid 263 in the third distillation column 230 may generate a low boiling point impurity stream 278 which may include, for example, methanol and other low boiling point impurities. The low boiling point impurity stream 278 can exit a third distillation column top lights outlet 234. A bottom outlet fluid 264 containing, for example, acetone, heavy ketones formed by aldol condensation of aldehydes, and/or deprotonated impurities can exit a third distillation column bottom outlet 236. The bottom outlet fluid 264 may be recycled to the second distillation column 220 via the caustic inlet 222 to treat the second distillation column 220 with caustic.

As shown in FIG. 2A, the second feed stream 209 may be supplied directly to the second distillation column 220. As noted above, the second feed stream 209 may additionally and/or alternatively be provided to the system via alternative feed locations. FIGS. 2B and 2C show examples of an acetone purification system 200B and an acetone purification system 200C, each with alternate feed locations for the second feed stream 209. The components of the acetone purification system 200B and the acetone purification system 200C are otherwise arranged similarly to those in the acetone purification system 200A depicted in FIG. 2A and the discussion above applies to these systems. Similarly, the fluid streams in FIGS. 2B and 2C may be referred to using the same reference numerals as in FIG. 2A and the discussion above applies to these systems. The fluid streams and components may, however, have different contents and/or different compositions depending on, for example, the arrangement of components in the system, the specific operating parameters used in conjunction with the system, etc.

FIG. 2B is a schematic view of an acetone purification system 200B in which the second feed stream 209 may be supplied to the first distillation column 210 in a manner similar to that discussed above with reference to FIG. 1B. In this embodiment, the first distillation column 210 may include a first distillation column auxiliary inlet 218 and the second feed stream 209 may be provided directly to the first distillation column 210 via the first distillation column auxiliary inlet 218.

FIG. 2C is a schematic view of an acetone purification system 200C in which the second feed stream may be supplied to both the first distillation column 210 and the second distillation column 220 in a manner similar to that discussed above with reference to FIG. 1C. As depicted in FIG. 2C, a first portion of second feed stream 209A can be provided to the second distillation column 220 via the second distillation column auxiliary inlet 228, and a second portion of second feed stream 209B can be provided to the first distillation column 210 via the first distillation column auxiliary inlet 218.

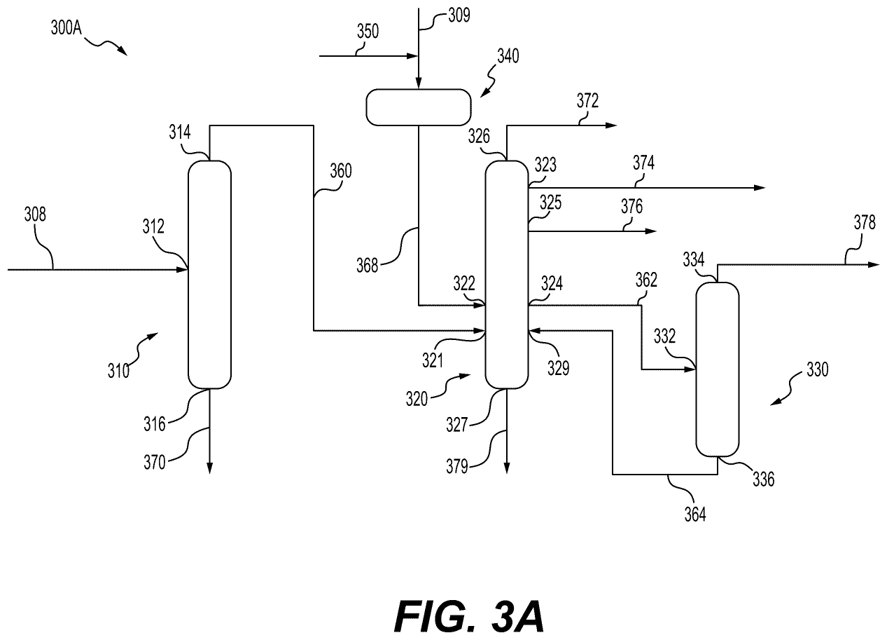

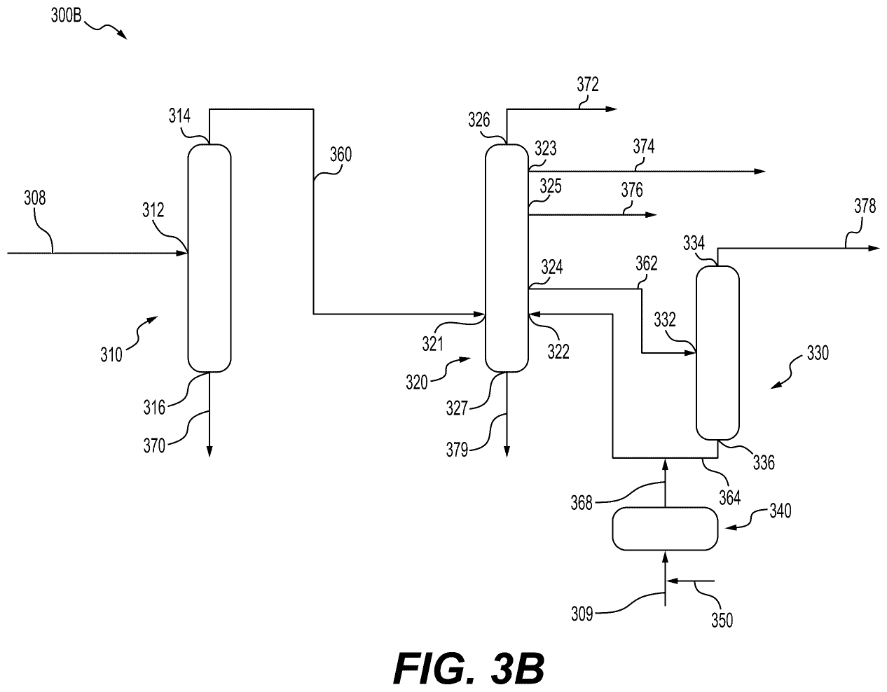

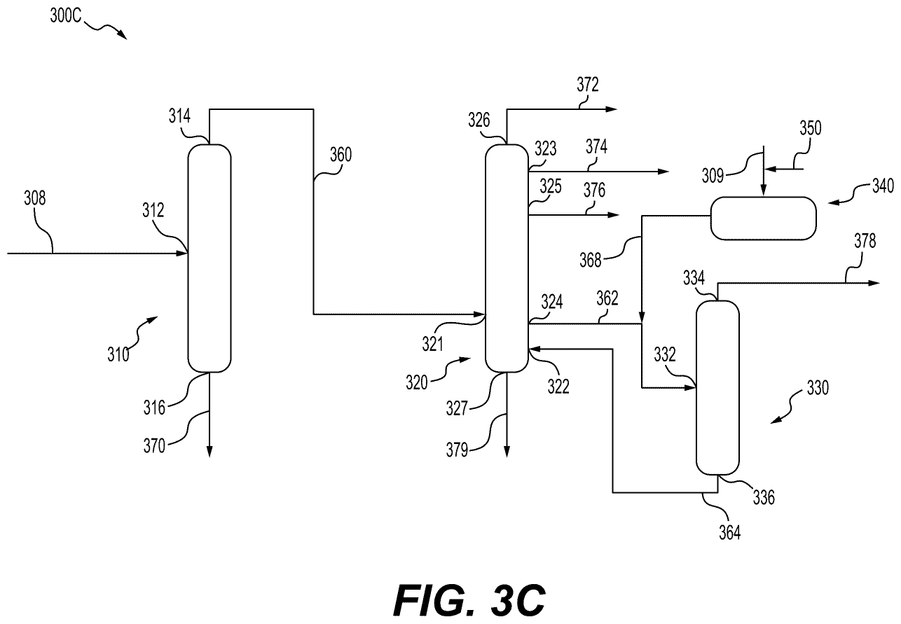

FIGS. 3A to 3C are schematic views of acetone purification systems for purifying acetone from a first feed stream and a second feed stream. In FIGS. 3A to 3C the second feed stream 309 may be fed directly to the caustic treatment vessel 340 instead of the first or second distillation column. For example, the caustic treatment vessel 340 may be in fluid communication with the caustic inlet 322. Elements of FIGS. 3A to 3C with reference numerals in the 300-series not described below are identical to those corresponding elements in FIGS. 1A to 1C, with otherwise identical reference numerals in the 100-series except the fluid streams and components may have different contents and/or different compositions depending on, for example, the arrangement of components in the system, the specific operating parameters used in conjunction with the system, etc.

FIG. 3A shows an acetone purification system 300A with the second feed stream 309 being caustic treated and supplied to a first location. In FIG. 3A, the caustic treatment vessel 340 may be upstream of the caustic inlet 322. The second feed stream 309 may undergo caustic treatment in the caustic treatment vessel 340 to generate a caustic treated stream 368 that may include, for example, acetone, phenate, and heavy ketones formed by aldol condensation of aldehydes. The caustic treated stream 368 may then be introduced into the second distillation column 320. Treating the second feed stream 309 in the caustic treatment vessel 340 before introducing the second feed stream 309 to the distillation column may be beneficial to more easily remove heavy ketones by aldol condensation of aldehydes.

In acetone purification system 300A, the second distillation column 320 may be in fluid communication with the third distillation column 330 without a caustic treatment vessel interposed between the second distillation column 320 and the third distillation column 330. This configuration may provide additional separation of low boiling point impurities from the crude stream 362 by distillation in the third distillation column 330. The bottom outlet fluid 364 may contain, for example, acetone and high boiling point impurities. The bottom outlet fluid 364 may be returned to the second distillation column 220 through a return inlet 329 for separation in the second distillation column 220.

FIG. 3B shows an alternative arrangement for the caustic treatment vessel 340. FIG. 3B shows an acetone purification system 300B with the second feed stream 309 being caustic treated and supplied to a second location. The second feed stream 309 may undergo caustic treatment in the caustic treatment vessel 340 to generate a caustic treated stream 368 that may include, for example, acetone, phenate, and heavy ketones formed by aldol condensation of aldehydes. The caustic treatment vessel 340 may output the caustic treated stream 368 downstream of the third distillation column bottom outlet 336 by adding the caustic treated stream 368 to the bottom outlet fluid 364. The caustic treated stream 368 may then be introduced into the second distillation column 320 via the caustic inlet 322. Treating the second feed stream 309 in the caustic treatment vessel 340 before introducing the second feed stream 309 to the distillation column may be beneficial to more easily remove heavy ketones by aldol condensation of aldehydes.

FIG. 3C shows an alternative arrangement for the caustic treatment vessel 340. FIG. 3C shows an acetone purification system 300C with the second feed stream 309 being caustic treated and supplied to a third location. The second feed stream 309 may undergo caustic treatment in the caustic treatment vessel 340 to generate a caustic treated stream 368 that may include, for example, acetone, phenate, and heavy ketones formed by aldol condensation of aldehydes. The caustic treatment vessel 340 may output the caustic treated stream 368 downstream of the side-draw outlet 324 by adding the caustic treated stream 368 to the crude stream 362. The caustic treated stream 368 may then be introduced into the third distillation column 330 via the third distillation column inlet 332. Treating the second feed stream 309 in the caustic treatment vessel 340 before introducing the second feed stream 309 to the third distillation column 330 may be beneficial to more easily remove heavy ketones by aldol condensation of aldehydes.

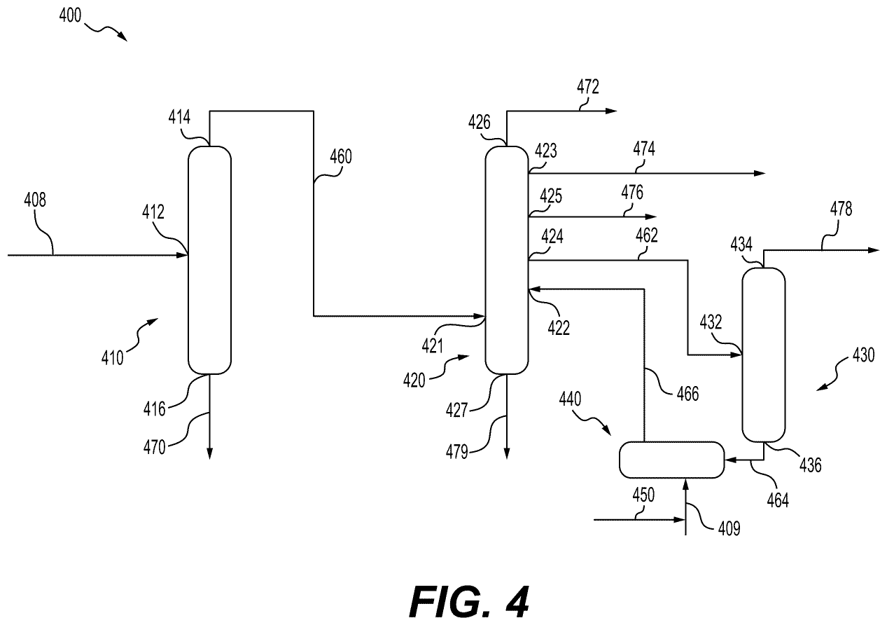

FIG. 4 is a schematic view of an acetone purification system 400 for purifying acetone from a first feed stream 408 and a second feed stream 409 in which the second feed stream 409 may be provided directly to a caustic treatment vessel 440 positioned between the second distillation column and the third distillation column as previously discussed with reference to FIGS. 1A to 1C. Elements of FIG. 4 with reference numerals in the 400-series, but not described below, are identical to those corresponding elements in FIGS. 1A to 1C, with otherwise identical reference numerals in the 100-series except the fluid streams and components may have different contents and/or different compositions depending on, for example, the arrangement of components in the system, the specific operating parameters used in conjunction with the system, etc. As above, treating the second feed stream 409 in the caustic treatment vessel 440 before introducing the second feed stream 409 to the distillation column may be beneficial to more easily remove heavy ketones by aldol condensation of aldehydes.

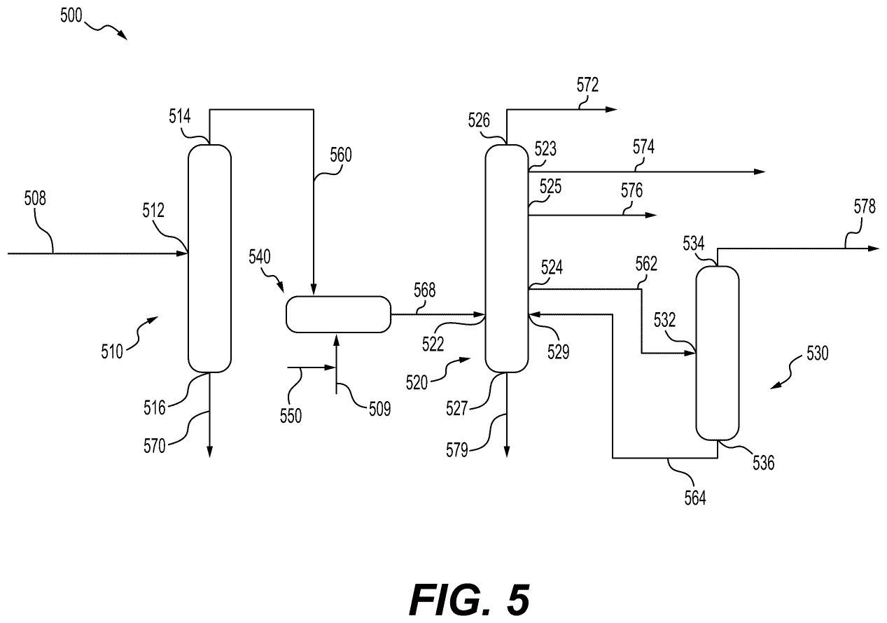

FIG. 5 is a schematic view of an acetone purification system 500 for purifying acetone from a first feed stream 508 and a second feed stream 509 in accordance with an embodiment. Elements of FIG. 5 with reference numerals in the 500-series, but not described below, are identical to those corresponding elements in FIGS. 1A to 1C, with otherwise identical reference numerals in the 100-series except the fluid streams and components may have different contents and/or different compositions depending on, for example, the arrangement of components in the system, the specific operating parameters used in conjunction with the system, etc.

In FIG. 5, a caustic treatment vessel 540 is positioned in fluid communication between the first distillation column top outlet 514 and the second distillation column acetone caustic inlet 522. The second feed stream 509 may undergo caustic treatment in the caustic treatment vessel 540 to generate a caustic treated stream 568 that may include, for example, acetone, phenate, and heavy ketones formed by aldol condensation of aldehydes. The caustic treated stream 568 may then be introduced into the second distillation column 520 via the caustic inlet 522. Treating the second feed stream 509 in the caustic treatment vessel 540 before introducing the second feed stream 509 to the second distillation column 520 may be beneficial to more easily remove heavy ketones by aldol condensation of aldehydes. For example, in some embodiments where the acetone enriched outflow 560 exits from the first distillation column top outlet 514 and is treated with caustic in the caustic treatment vessel 540 prior to distilling the caustic treated stream 568 in the second distillation column 520, the purified acetone stream 574 can have a total aldehyde content ranging from twenty weight percent to sixty weight percent by total weight of the purified acetone stream 574.

FIGS. 1A to 5 discussed above provide non-limiting example configurations of the system for purifying acetone (i.e., an acetone purification system). The features depicted FIGS. 1A to 5 can be independently chosen and/or combined to form the system for purifying acetone. For example, the second feed stream can be split into various portions (e.g., a first portion, a second portion, etc.). The second feed stream or its various portions can be provided directly to any location chosen from, for example, a first column, a second column, a caustic treatment vessel, and combinations thereof. For example, the second feed stream can be provided directly to a first column, a second column, or a caustic treatment vessel. The second feed stream can split into two portions and provided directly to a first column and a second column, a first column and a caustic treatment vessel, or a caustic treatment vessel and a second column. The second feed stream can split into three portions and provided directly to a first column, a second column, and a caustic treatment vessel.

As discussed above, the inventor discovered that the cumene hydroperoxide cleavage product and the acetone stream from a bisphenol A production plant can be co-purified using the systems and methods disclosed herein with synergistic and unexpected benefits. These synergistic benefits include, for example, reduced energy expenditure to purify acetone from the feed streams and improved purity of the product acetone stream.

The disclosed acetone purification system described with reference to FIGS. 1A to 5, may be useful in methods of purify acetone from the first feed stream 108 and the second feed stream 109.

A method of purifying acetone from the first feed stream and the second feed stream using the disclosed systems for purifying acetone may include providing the first feed stream directly to the first feed stream inlet and providing the second feed stream directly to the first distillation column auxiliary inlet, providing the second feed stream directly to the second distillation column auxiliary inlet, and/or providing the second feed stream directly to the caustic treatment vessel. In some embodiments, the second feed stream may be provided directly to the first distillation column auxiliary inlet. In some embodiments, the second feed stream may be provided directly to the second distillation column auxiliary inlet. In some embodiments, the second feed stream may be provided directly to the caustic treatment vessel.

Disclosed are methods of purifying acetone from the first feed stream and the second feed stream. In embodiments, a method of purifying acetone from the first feed stream and the second feed stream may include distilling the first feed stream in the first distillation column such that a phenol enriched outflow exits from the first distillation column bottom outlet and such that an acetone enriched outflow exits from the first distillation column top outlet.

The method may include distilling the acetone enriched outflow in the second distillation column in the presence of a caustic fluid. The distillation in the second distillation column can cause low boiling point impurities (e.g., methanol) to exit out of the second distillation column top lights outlet. The distillation in the second distillation column can cause a purified acetone stream to exit out of the acetone product outlet positioned below the second distillation column top lights outlet. In some embodiments, the purified acetone stream has a total aldehyde content ranging from twenty weight percent to one hundred weight percent by total weight of the purified acetone stream. The distillation in the second distillation column can cause an impure acetone exit stream to exit out of the impure acetone outlet positioned below the acetone product outlet. The distillation in the second distillation column can cause the crude stream to exit out of the side-draw outlet positioned below the impure acetone outlet.

The method may include providing the crude stream out of the side-draw outlet and into the third distillation column. In some embodiments, the crude stream may be contacted with caustic to form a caustic treated crude stream (referred to herein as a caustic treated fluid) which may be then provided to the third distillation column. Distillation in the third distillation column can cause low boiling point impurities (e.g., methanol) to exit out of the third distillation column top lights outlet. Distillation in the third distillation column 130 can cause a bottom outlet fluid 164 to exit out of the third distillation column bottom outlet. In some embodiments, the bottom outlet fluid may be contacted with caustic to form a caustic treated fluid and the caustic treated fluid can be provided to the second distillation column. In some embodiments, the bottom outlet fluid may exit out of the third distillation column bottom outlet to be provided directly to the second distillation column.

The method may include providing the second feed stream directly to the first distillation column, providing the second feed stream directly to the second distillation column, or caustic treating the second feed stream to form a caustic treated second feed stream and providing the caustic treated second feed stream directly to the second distillation column. In some embodiments, the second feed stream may be provided directly to the first distillation column. In some embodiments, the second feed stream may be provided directly to the second distillation column. In some embodiments, the second feed stream may be caustic treated to form the caustic treated second feed stream and the caustic treated second feed stream may be provided directly to the second distillation column.

As discussed above, the inventor discovered that the cumene hydroperoxide cleavage product and the acetone stream from a bisphenol A production plant can be co-purified using the systems and methods disclosed herein with synergistic and unexpected benefits. These synergistic benefits include, for example, reduced energy expenditure to purify acetone from the feed streams and improved purity of the product acetone stream.

Further aspects of the present disclosure are provided by the subject matter of the following clauses.

A system for purifying acetone from a first feed stream including acetone, cumene, phenol, alpha methyl styrene, water and methanol, and a second feed stream rich in acetone and including aldehydes and methanol. The system includes a first distillation column including a first feed stream inlet to receive the first feed stream, a first distillation column top outlet to output an acetone enriched fluid, and a first distillation column bottom outlet to output a phenol enriched outflow. The system includes a second distillation column including a second distillation column acetone enriched inlet in fluid communication with the first distillation column top outlet, a caustic inlet to treat contents of the second distillation column with caustic, the caustic inlet being positioned above the second distillation column acetone enriched inlet, an acetone product outlet to output purified acetone, the acetone product outlet being positioned above the caustic inlet, a side-draw outlet positioned below the acetone product outlet, a second distillation column top lights outlet to output a first volatile impurity, the second distillation column top lights outlet being positioned above the acetone product outlet, and a second distillation column bottom outlet to output phenate. The system includes a third distillation column including a third distillation column inlet in fluid communication with the side-draw outlet, a third distillation column top lights outlet to output a second volatile impurity, and a third distillation column bottom outlet. The system includes a caustic treatment vessel in fluid communication between the first distillation column top outlet and the caustic inlet, in fluid communication between the second feed stream and the second distillation column, in fluid communication between the side-draw outlet and the third distillation column inlet, or in fluid communication between the third distillation column bottom outlet and the caustic inlet of the second distillation column. In the system, the first distillation column includes a first distillation column auxiliary inlet in direct fluid communication with the second feed stream, the second distillation column includes a second distillation column auxiliary inlet in fluid communication with the second feed stream, and/or the caustic treatment vessel is in fluid communication between the caustic inlet and the second feed stream.

The system of the preceding clause such that the first distillation column includes the first distillation column auxiliary inlet in direct fluid communication with the second feed stream.

The system of any of the preceding clauses such that the system includes an impure acetone outlet positioned above the side-draw outlet and below the acetone product outlet.

The system of any of the preceding clauses such that the system includes the caustic treatment vessel in fluid communication between the third distillation column bottom outlet and the caustic inlet of the second distillation column.

The system of any of the preceding clauses such that the second distillation column includes the second distillation column auxiliary inlet in fluid communication with the second feed stream.

The system of any of the preceding clauses such that the first distillation column includes the first distillation column auxiliary inlet in direct fluid communication with the second feed stream, and the second distillation column includes the second distillation column auxiliary inlet in direct fluid communication with the second feed stream.

The system of any of the preceding clauses such that the caustic treatment vessel is in fluid communication between the caustic inlet and the second feed stream.

The system of any of the preceding clauses such that the caustic treatment vessel is in fluid communication between the second feed stream and the second distillation column.

The system of any of the preceding clauses such that the caustic treatment vessel is in fluid communication between the caustic inlet and the second feed stream.

The system of any of the preceding clauses such that the caustic treatment vessel is in fluid communication between the second feed stream and the second distillation column, and the caustic treatment vessel is in fluid communication between the caustic inlet and the second feed stream.

The system of any of the preceding clauses such that the caustic treatment vessel is in fluid communication between the second distillation column and the third distillation column.

The system of any of the preceding clauses such that the caustic treatment vessel is in fluid communication between the side-draw outlet of the second distillation column and the inlet of the third distillation column.

The system of any of the preceding clauses such that the caustic treatment vessel is in fluid communication between the side-draw outlet and the third distillation column inlet, and the first distillation column includes the first distillation column auxiliary inlet in direct fluid communication with the second feed stream.

The system of any of the preceding clauses such that the caustic treatment vessel is in fluid communication between the side-draw outlet and the third distillation column inlet, and the second distillation column includes the second distillation column auxiliary inlet in direct fluid communication with the second feed stream.

The system of any of the preceding clauses such that the caustic treatment vessel is in fluid communication between the third distillation column bottom outlet and the caustic inlet.

The system of any of the preceding clauses such that the caustic treatment vessel is in fluid communication between the third distillation column bottom outlet and the caustic inlet, and the caustic treatment vessel is in fluid communication between the second feed stream and the second distillation column.

The system of any of the preceding clauses such that the caustic treatment vessel is in fluid communication between the third distillation column bottom outlet and the caustic inlet, and the first distillation column includes the first distillation column auxiliary inlet in direct fluid communication with the second feed stream.

The system of any of the preceding clauses such that the caustic treatment vessel is in fluid communication between the third distillation column bottom outlet and the caustic inlet, and the second distillation column includes the second distillation column auxiliary inlet in direct fluid communication with the second feed stream.

The system of any of the preceding clauses such that the third distillation column bottom outlet is in direct fluid communication with the caustic inlet.

The system of any of the preceding clauses such that the third distillation column inlet is in direct fluid communication with the side-draw outlet.

The system of any of the preceding clauses such that the second distillation column acetone enriched inlet is in direct fluid communication with the first distillation column top outlet.

The system of any of the preceding clauses such that the caustic treatment vessel is in direct fluid communication between the second feed stream and the second distillation column.

The system of any of the preceding clauses such that the caustic treatment vessel is direct fluid communication between the side-draw outlet and the third distillation column.

The system of any of the preceding clauses, such that the caustic treatment vessel provides a residence time of three minutes to sixty minutes.

The system of any of the preceding clauses, such that the caustic treatment vessel is free from internal structures.

The system of any of the preceding clauses, such that the caustic treatment vessel comprises one or more of static mixing internals, an agitator, or a circulation pump.

The system of any of the preceding clauses, such that the caustic treatment vessel has a temperature of forty-five degrees Celsius to seventy-five degrees Celsius.

The system of any of the preceding clauses, such that the purified acetone has a total concentration of aldehydes less than one hundred fifty parts per million by weight.

The system of any of the preceding clauses, such that the purified acetone has a total concentration of aldehydes ranging from twenty parts per million by weight to one hundred parts per million by weight.

The system of any of the preceding clauses, such that the purified acetone has a methanol content of less than two hundred fifty parts per million by weight.

The system of any of the preceding clauses, such that the purified acetone has a methanol content ranging from fifty parts per million by weight to one hundred parts per million by weight.

The system of any of the preceding clauses, such that the caustic treatment vessel is in fluid communication between the first distillation column top outlet and the caustic inlet.

A method of purifying acetone from a first feed stream including acetone, cumene, phenol, alpha methyl styrene, water and methanol and a second feed stream rich in acetone and including aldehydes and methanol with the system of any of the preceding clauses. The method includes providing the first feed stream directly to the first distillation column inlet and providing the second feed stream directly to the first distillation column auxiliary inlet, providing the second feed stream directly to the second distillation column auxiliary inlet, and/or providing the second feed stream directly to the caustic treatment vessel, and separating acetone from methanol in the second distillation column to generate a purified acetone steam.

The method of the preceding clause such that the second feed stream is provided directly to the first distillation column auxiliary inlet.

The method of any of the preceding clauses such that the second feed stream is provided directly to the second distillation column auxiliary inlet.

The method of any of the preceding clauses such that the second feed stream is provided directly to the caustic treatment vessel.

The method of any of the preceding clauses such that the purified acetone steam exits the acetone product outlet.

The method of any of the preceding clauses such that the purified acetone steam having at least ninety-nine weight percent acetone by total weight of the purified acetone steam exits the acetone product outlet.

The method of any of the preceding clauses such that phenol exits the first distillation column bottom outlet.

The method of any of the preceding clauses such that sodium phenate exits the second distillation column bottom outlet.

The method of any of the preceding clauses such that methanol exits the second distillation column top lights outlet.

The method of any of the preceding clauses such that methanol exits the third distillation column top lights outlet.

The method of any of the preceding clauses such that caustic is fed into the caustic treatment vessel to maintain a caustic pH in the caustic treatment vessel.

The method of any of the preceding clauses such that an exit stream including acetone exits the impure acetone outlet.

The method of any of the preceding clauses such that an exit stream including acetone having a weight percentage of acetone ranging from one weight percent to ninety weight percent by total weight of the exit stream exits the impure acetone outlet.

The method of any of the preceding clauses such that the first feed stream is in fluid communication with a cumene hydroperoxide cleavage reactor.

The method of any of the preceding clauses such that the first feed stream is in fluid communication with a cumene hydroperoxide cleavage reactor, an exit stream including acetone exits the impure acetone outlet, and the exit stream including acetone is in fluid communication with the cumene hydroperoxide cleavage reactor.

The method of any of the preceding clauses such that the second feed stream is in fluid communication with a Bisphenol A formation reactor.

A method of purifying acetone from a first feed stream including acetone, cumene, phenol, alpha methyl styrene, water and methanol and a second feed stream rich in acetone and including aldehydes and methanol. The method includes distilling the first feed stream in a first distillation column such that a phenol enriched outflow exits from a first distillation column bottom outlet and such that an acetone enriched outflow exits from a first distillation column top outlet. The method includes distilling the acetone enriched outflow in the presence of a caustic fluid such that methanol exits out of a second distillation column top lights outlet, such that a purified acetone stream exits out of an acetone product outlet positioned below the second distillation column top lights outlet, such that an impure acetone stream exits out of an impure acetone outlet positioned below the acetone product outlet, and such that a crude stream exits out of a side-draw outlet positioned below the impure acetone outlet. The method includes distilling the crude stream in a third distillation column such that methanol exits out of a third distillation column top lights outlet and such that a bottom outlet fluid exits out of a third distillation column bottom outlet where the bottom outlet fluid is contacted with a caustic to form a caustic treated bottom outlet fluid, and the caustic treated bottom outlet fluid is provided to the second distillation column, or contacting the crude stream with a caustic to form a caustic treated crude stream and distilling the caustic treated crude stream in the third distillation column such that methanol exits out of the third distillation column top lights outlet, and such that the bottom outlet fluid exits out of the third distillation column bottom outlet wherein the bottom outlet fluid is provided to the second distillation column. The method includes providing the second feed stream directly to the first distillation column, providing the second feed stream directly to the second distillation column, or caustic treating the second feed stream to form a caustic treated second feed stream and providing the caustic treated second feed stream directly to the second distillation column.

The method of the preceding clause such that the second feed stream is provided directly to the first distillation column.

The method of any of the preceding clauses such that the second feed stream is provided directly to the second distillation column.

The method of any of the preceding clauses such that the second feed stream is caustic treated to form the caustic treated second feed stream and the caustic treated second feed stream is provided directly to the second distillation column.

The method of any of the preceding clauses such that the purified acetone stream has at least ninety-nine weight percent acetone by total weight of the purified acetone stream.

The method of any of the preceding clauses such that phenol exits the first distillation column bottom outlet.

The method of any of the preceding clauses such that sodium phenate exits the second distillation column bottom outlet.

The method of any of the preceding clauses such that methanol exits the second distillation column top lights outlet.

The method of any of the preceding clauses such that methanol exits the third distillation column top lights outlet.

The method of any of the preceding clauses such that caustic is fed into the caustic treatment vessel to maintain a caustic pH in the caustic treatment vessel.

The method of any of the preceding clauses such that the impure acetone stream has a weight percentage of acetone ranging from one weight percent to ninety weight percent by total weight of the impure acetone stream.

The method of any of the preceding clauses such that the first feed stream is provided from a cumene hydroperoxide cleavage reactor.

The method of any of the preceding clauses such that the first feed stream is provided from a cumene hydroperoxide cleavage reactor and the impure acetone stream is provided to the cumene hydroperoxide cleavage reactor.

The method of any of the preceding clauses such that the second feed stream is an acetone rich stream from a Bisphenol A production plant.

The method of any of the preceding clauses such that the crude stream includes from 0.1 weight percent acetone to one weight percent acetone by total weight of the crude stream.

The method of any of the preceding clauses such that the purified acetone stream has a total aldehyde content ranging from twenty weight percent to one hundred weight percent by total weight of the purified acetone stream.

The method of any of the preceding clauses such that the acetone enriched outflow exits from the first distillation column top outlet and is treated with caustic in a caustic treatment vessel prior to distilling the acetone enriched outflow in the second distillation column.

The method of any of the preceding clauses such that the purified acetone stream has a total aldehyde content ranging from twenty weight percent to sixty weight percent by total weight of the purified acetone stream.

Although the foregoing description is directed to some exemplary embodiments of the present disclosure, other variations and modifications will be apparent to those skilled in the art. Moreover, features described in connection with one embodiment of the present disclosure may be used in conjunction with other embodiments, even if not explicitly stated above.

1. A system for purifying acetone from a first feed stream comprising acetone, cumene, phenol, alpha methyl styrene, aldehydes, water, and methanol, and a second feed stream rich in acetone and including aldehydes and methanol, the system comprising:
a first distillation column including a first feed stream inlet to receive the first feed stream, a first distillation column top outlet to output an acetone enriched fluid, and a first distillation column bottom outlet to output a phenol enriched outflow;
a second distillation column including a second distillation column acetone enriched inlet in fluid communication with the first distillation column top outlet, a caustic inlet to treat contents of the second distillation column with caustic, the caustic inlet being positioned above the second distillation column acetone enriched inlet, an acetone product outlet to output purified acetone, the acetone product outlet being positioned above the caustic inlet, a side-draw outlet positioned below the acetone product outlet, a second distillation column top lights outlet to output a first volatile impurity, the second distillation column top lights outlet being positioned above the acetone product outlet, and a second distillation column bottom outlet to output phenate;
a third distillation column including a third distillation column inlet in fluid communication with the side-draw outlet, a third distillation column top lights outlet to output a second volatile impurity, and a third distillation column bottom outlet; and
a caustic treatment vessel in fluid communication between the first distillation column top outlet and the caustic inlet, in fluid communication between the second feed stream and the second distillation column, in fluid communication between the side-draw outlet and the third distillation column inlet, or in fluid communication between the third distillation column bottom outlet and the caustic inlet of the second distillation column,
wherein the first distillation column includes a first distillation column auxiliary inlet in direct fluid communication with the second feed stream, the second distillation column includes a second distillation column auxiliary inlet in fluid communication with the second feed stream, and/or the caustic treatment vessel is in fluid communication between the caustic inlet and the second feed stream.

2. The system of claim 1, wherein the first distillation column includes the first distillation column auxiliary inlet in direct fluid communication with the second feed stream, and wherein the second distillation column includes the second distillation column auxiliary inlet in direct fluid communication with the second feed stream.

3. The system of claim 1, wherein the caustic treatment vessel is in fluid communication between the caustic inlet and the second feed stream, between the second feed stream and the second distillation column, between the second distillation column and the third distillation column, or between the side-draw outlet of the second distillation column and the third distillation column inlet.

4. The system of claim 1, wherein the caustic treatment vessel is in fluid communication between the second feed stream and the second distillation column, and the caustic treatment vessel is in fluid communication between the caustic inlet and the second feed stream.

5. The system of claim 1, wherein the caustic treatment vessel is in fluid communication between the side-draw outlet and the third distillation column inlet, and the first distillation column includes the first distillation column auxiliary inlet in direct fluid communication with the second feed stream.

6. The system of claim 1, wherein the caustic treatment vessel is in fluid communication between the side-draw outlet and the third distillation column inlet, and the second distillation column the second distillation column auxiliary inlet in direct fluid communication with the second feed stream.

7. The system of claim 1, wherein the caustic treatment vessel is in fluid communication between the third distillation column bottom outlet and the caustic inlet.

8. The system of claim 1, wherein the caustic treatment vessel is in fluid communication between the third distillation column bottom outlet and the caustic inlet, and the caustic treatment vessel is in fluid communication between the second feed stream and the second distillation column.

9. The system of claim 1, wherein the caustic treatment vessel is in fluid communication between the third distillation column bottom outlet and the caustic inlet, and the first distillation column includes the first distillation column auxiliary inlet in direct fluid communication with the second feed stream.

10. The system of claim 1, wherein the caustic treatment vessel is in fluid communication between the third distillation column bottom outlet and the caustic inlet, and the second distillation column includes the second distillation column auxiliary inlet in direct fluid communication with the second feed stream.

11. The system of claim 1, wherein the third distillation column bottom outlet is in direct fluid communication with the caustic inlet.

12. The system of claim 1, wherein the third distillation column inlet is in direct fluid communication with the side-draw outlet.

13. The system of claim 1, wherein the second distillation column acetone enriched inlet is in direct fluid communication with the first distillation column top outlet.

14. The system of claim 1, wherein the caustic treatment vessel is in direct fluid communication between the second feed stream and the second distillation column.

15. The system of claim 1, wherein the caustic treatment vessel is direct fluid communication between the side-draw outlet and the third distillation column.

16. The system of claim 1, wherein the caustic treatment vessel is free from internal structures.

17. The system of claim 1, wherein the caustic treatment vessel comprises one or more of static mixing internals, an agitator, or a circulation pump.

18. The system of claim 1, wherein the caustic treatment vessel is in fluid communication between the first distillation column top outlet and the caustic inlet.

19. A method of purifying acetone from a first feed stream including acetone, cumene, phenol, alpha methyl styrene, water and methanol and a second feed stream rich in acetone and including aldehydes and methanol, the method comprising:
distilling the first feed stream in a first distillation column such that a phenol enriched outflow exits from a first distillation column bottom outlet and such that an acetone enriched outflow exits from a first distillation column top outlet;
distilling the acetone enriched outflow in the presence of a caustic fluid such that methanol exits out of a second distillation column top lights outlet, such that a purified acetone stream exits out of an acetone product outlet positioned below the second distillation column top lights outlet, such that an impure acetone stream exits out of an impure acetone outlet positioned below the acetone product outlet, and such that a crude stream exits out of a side-draw outlet positioned below the impure acetone outlet;
distilling the crude stream in a third distillation column such that methanol exits out of a third distillation column top lights outlet and such that a bottom outlet fluid exits out of a third distillation column bottom outlet where the bottom outlet fluid is contacted with a caustic to form a caustic treated bottom outlet fluid, and the caustic treated bottom outlet fluid is provided to the second distillation column, or contacting the crude stream with a caustic to form a caustic treated crude stream and distilling the caustic treated crude stream in the third distillation column such that methanol exits out of the third distillation column top lights outlet, and such that the bottom outlet fluid exits out of the third distillation column bottom outlet wherein the bottom outlet fluid is provided to the second distillation column; and
providing the second feed stream directly to the first distillation column, providing the second feed stream directly to the second distillation column, or caustic treating the second feed stream to form a caustic treated second feed stream and providing the caustic treated second feed stream directly to the second distillation column.

20. The method of claim 19, wherein the second feed stream is provided directly to the first distillation column.

21. The method of claim 19, wherein the second feed stream is provided directly to the second distillation column.

22. The method of claim 19, wherein the second feed stream is caustic treated to form the caustic treated second feed stream and the caustic treated second feed stream is provided directly to the second distillation column.

23. The method of claim 19, wherein the impure acetone stream has a weight percentage of acetone ranging from one weight percent to ninety weight percent by total weight of the impure acetone stream.

24. The method of claim 19, wherein the first feed stream is provided from a cumene hydroperoxide cleavage reactor.

25. The method of claim 19, wherein the second feed stream is an acetone rich stream from a Bisphenol A production plant.

26. The method of claim 19, wherein the crude stream comprises from 0.1 weight percent acetone to one weight percent acetone by total weight of the crude stream.
